SEQUENCE LISTING (4 sequences)

SEQ ID NO: 1 - protein, 6 aa, Artificial Sequence
  synthetic oligopeptide which acts as HIV-integrase inhibitor
HCKFWW

SEQ ID NO: 2 - protein, 15 aa, Artificial Sequence
  oligopeptide derived from BSA
RPCFSALTPDEYVPK

SEQ ID NO: 3 - protein, 7 aa, Artificial Sequence
  oligopeptide derived from BSA
LCVLHEK

SEQ ID NO: 4 - protein, 5 aa, Artificial Sequence
  oligopeptide derived from BSA
CDEWQ

(19) United States
(12) Patent Application Publication
(10) Pub. No.: US 2010/0167262 A1
(43) Pub. Date: Jul. 1, 2010

(54) METHOD AND REAGENT FOR THE SPECIFIC IDENTIFICATION AND QUANTIFICATION OF ONE OR MORE PROTEINS IN A SAMPLE USING IN PARTICULAR INDUCTIVELY COUPLED PLASMA-MASS SPECTROMETRY

(75) Inventors: Michael Linscheid, Berlin (DE); Stefan Pieper, Berlin (DE); Christian Scheler, Berlin (DE); Robert Ahrends, Berlin (DE)

Correspondence Address: ROTHWELL, FIGG, ERNST & MANBECK, P.C., 1425 K STREET, N.W., SUITE 800, WASHINGTON, DC, 20005, US

(73) Assignees: Proteome Factory AG, Berlin (DE); Humboldt-Universitaet ZU Berlin, Berlin (DE)

(21) Appl. No.: 12/297,092

(22) PCT Filed: Apr. 13, 2007
(86) PCT No.: PCT/EP2007/003419

§ 371 Date: Jun. 19, 2009

(30) Foreign Application Priority Data
Apr. 14, 2006 (EP) 06007919.1

(51) Int. Cl. C12Q 1/00; G01N 33/00

(52) U.S. Cl. 435/4; 436/86

(57) ABSTRACT

The invention relates to a method for the identification and quantification of one or more biomolecules in a sample containing a mixture of substances, wherein said method comprises the steps of, a) providing a sample which contains one or more biomolecules, b) providing a reagent for the analysis of biomolecules according to Formula I (A-Y-PRG) wherein, A may be present or absent and when A is present, A is H or constitutes at least one functional group for the reversible, covalent or non-covalent binding to a support material, whereby Y is a group comprising at least one chelate function for metals, preferable low in isotopes, and whereby PRG is a reactive group for the selective binding of biomolecules to be analyzed, c) coupling the one or more biomolecules from step a) to the reagent of step b), d) selecting the one or more biomolecules labeled in step c) under the employment of a functional group for the reversible, covalent or non-covalent binding to a support material and removal of the one or more unbound biomolecules, wherein (d) optionally comprises releasing the bound biomolecules from the support material and the elution from the matrix; and f) detecting and identifying the one or more labeled biomolecules by means of Inductively Coupled Plasma-Mass Spectrometry (ICP-MS), wherein f) optionally comprises cleaving the one or more biomolecules in said sample wherein, said cleaving may be performed (i) before coupling of the reagent PRG or (ii) after coupling.

### FIELD OF THE INVENTION

The invention relates to the field of molecular biology. In particular the invention is in the field of proteomics. In one aspect the invention relates to analytical testing. In particular the invention relates to a method and a reagent suitable for performing said method, whereby the method allows for reproducible, systematic, qualitative and quantitative proteome and/or protein characterization by means of non-isotope metal coded markers and Inductively Coupled Plasma-Mass Spectrometry (IC PMS).

### BACKGROUND OF THE INVENTION

As a scientific field, proteome research (“Proteomics”) deals with the systematic identification of all proteins being expressed within a cell or tissue, and with the characterization of their essential features like e.g. amount, degree of modification, integration into multi-protein complexes, etc. Protein databases or cell maps are created, which serve the archiving of the protein sequences.

Herein, the following abbreviations are used, 2-D (Two dimensional), CHAPS (3-(3-Cholamidopropyl)dimethylammonio-1-propane sulfate), DPAGE (Dissolvable polyacrylamide gel electrophoresis), DTT (Dithiothreitol), EDTA (Ethylenediamminetetraacetate) ESI (Electrospray ionization), EST (Expressed sequence tags (cDNA)) FIA (Flow injection analysis), HEPES (4-(2-Hydroxyethyl)-1-piperazineethanesulfonic acid), ICP MS or ICPMS (Inductively coupled plasma-mass spectrometry), IEF (Isoelectric focusing) LC (Liquid chromatography), MALDI (Matrix assisted laser desorption ionisation) MeCAT (Metal coded affinity tag) MS (Mass spectrometry) NCBI (National center for biotechnology information) PA (Polyacrylamid), ppb (Parts per billion (ng/ml)), ppq (Parts per quadrillion (fg/ml)), SDS-PAGE (Sodium dodecylsulfate-polyacrylamide gel electrophoresis), Ss (Sus scrofa), TCEP (Tris(2-carboxyethyl)phosphine hydrochlorid) and ToF (Time of flight).

Currently, many thousands of sequences and often also their investigated functions are available.

At present however, none of the applied analytical protein technologies reaches the high throughput and the level of automation of genetic engineering. Moreover, it is improbable, that a protein amplification technique analogous e.g. to PCR will be ever realizable in proteome research. What seems to be more appropriate rather is the possibility of protein enrichment, wherein the proteins of interest are extracted or enriched according to specific characteristics. It is e.g. possible to make use of physical characteristics like solubility or the capability to bind to specific ligands.

The method of proteome analysis, which is still employed most and is most reliable, is the two-dimensional gel electrophoresis (2DGE), which is subsequently followed by the sequence identification of the separated protein species. This approach reached its scientific importance for reason of the enormous progress in mass spectrometry and bioinformatics. This MS-technique, which has just recently become available and is highly sensitive, has made it possible to detect even minimal amounts of proteins and peptides—which can be made visible by conventional staining techniques—in the range of femtomoles and moreover to identify them by tandem techniques. These techniques in particular are the matrix assisted laser desorption/ionisation (MALDI) time-of-flight (TOF)-MS and the electrospray ionisation (ESI)-MS. Tandem-MS instruments like the triple-quadrupole device, ion trap, and the hybrid quadrupole time-of-flight (Q-TOF) device are routinely employed in LC-MS/MS- or nanospray experiments with electrospray ionisation (ESI), in order to generate peptide fragment ion spectra, which are suitable for the protein identification via a database sequence search.

The protein or genome data base search constitutes a tool of equal importance, which has largely contributed to the progress of proteome research. The computer search algorithms, which have been developed, are very sophisticated nowadays. Goodlett et al. were able to show that the exact mass of a peptide, in combination with limiting search criteria like the molecular weight of the protein the peptide is derived from, and the indication of the specific protease for cleaving the protein, can be sufficient for an unambiguous identification of a protein in a data base search. The high expenditure of work and the often observed non-reproducibility of the 2DGE-technique between different laboratories however make it nearly impossible to automate this method. Nowadays, there exists no analytical technique in the field of proteome research, which reaches a level comparable to genome technology. Although one is able to analyze the components of a protein mixture by means of these methods, they are neither suitable to determine the exact quantity nor the state of activity of the proteins in the mixture. Without a previous enrichment step it is virtually impossible to detect proteins being present only in very low quantities, like e.g. regulator proteins. For this reason and because of further known drawbacks of the 2DGE, research is increasingly focused on alternative methods in order to become largely independent from the 2DGE as a separation method.

Gel-free systems find the increasing interest of the proteome researchers. One can image various different gel-free systems, which are all based on the combination of two or more different chromatographic separation methods. The chromatographic separation of proteins is an essential element in any protein research and thus also constitutes an obvious method in proteome research. Due to long-standing development and optimization, chromatographic separation allows for a high reproducibility. However, even the combination of two different chromatographic methods will not allow for the resolution required in proteome research, since complex protein mixtures for reason of their characteristics are hardly to be separated into their individual, purified protein fractions. A combination between chromatography and mass spectrometry offers a further dimension of separation, the mass spectrometry, but will—when being applied to proteins—only have a very limited use advantage. As it is described in the following, this approach however will not promise success before peptides can be analyzed.

WO 00/11208 discloses an interesting alternative method for proteome analysis, which is particularly suitable for the quantitative analysis of protein expression in complex biological samples like cells and tissues, for the detection and quantification of specific proteins in complex protein mixtures and also for the quantitative determination of specific enzyme activity. It makes use of a novel class of chemical reagents, the coded affinity tags (CATs), in this case the so-called isotope-coded affinity tags (ICATs) and methods of mass spectrometry.

The ICAT-reagent consists of the affinity tag, which selectively binds to a corresponding counter-reagent in a non-covalent manner and thus allows for the separation of the affinity tag-labeled peptides or substrates from the remaining mixture by means of column chromatography. The affinity tag is coupled to a reactive group via a linker, which may optionally carry an isotope label, wherein the reactive group selectively reacts with a specific protein function.

In this way, the proteins, after being isolated from the cells, are labeled by the ICAT-reagent at specific binding sites. Here, one may e.g. have a functional group showing a specific reactivity for sulfhydryl groups, thus exclusively binding to proteins containing cysteine. From the peptide mixture obtained after enzymatic hydrolysis, one consequently isolates only the cysteine-containing peptides in a selective manner. This allows for a significant reduction of complexity of the obtained peptide mixture, since less than a tenth of all peptides, which are e.g. released from the entire yeast proteome by tryptic cleavage contain a cysteine-containing residue. A further significant advantage is that one by this approach can enrich proteins only being present in minor amounts. Despite the significant reduction of the system's complexity however, the quantification and identification of the proteins is ensured.

In order to quantitatively detect the relative amount of proteins in one or more protein samples, one now uses ICAT-reagents being isotope-coded in a different manner. Each sample is treated with an ICAT-reagent, which carries different isotope labeling, but is otherwise chemically identical. The samples, which may e.g. be derived from cell cultures of a species in different developmental phases, are unified in the following and enzymatically hydrolyzed. The labeled peptides are separated from the mixture by affinity chromatography and then fractionated by means of HPLC. A pair of peptides being identical, but being derived from different samples, is simultaneously eluted from the HPLC-column. In the mass spectrum, these peptides however do not display the same mass/charge-ratio, but differ by the characteristic mass difference of the differently isotope-labeled tags. The ratio of the relative ionic intensities of such a (CAT-labeled) peptide pair in the mass spectrum quantitatively mirrors the relative quantitative proportion of the basal proteins in the cells or tissue of origin. The peptide sequence of the ICAT-labeled peptide is then determined by fragmentation using a tandem mass spectrometer (MS/MS). The protein identification is then accomplished by a computer-aided genome or protein data base search on the basis of the recorded sequence information.

Despite all major advantages of the ICAT-method, there are still several disadvantages in its performance, which impede and complicate its use in the field of high throughput analysis. One has to employ isotopes, which significantly raise the synthesis costs of the compounds and only are of limited availability at affordable prices, thereby further restricting the method's flexibility.

It is thus the object of the present invention to provide an improved CAT-based method allowing for the employment of CAT under high throughput conditions. It is a further object to provide a CAT-reagent being specifically suitable for this method.

According to a first aspect of the present invention, this object is achieved by a method for the identification and quantification of one or more proteins in a sample containing

### DESCRIPTION OF THE INVENTION

The invention relates to a method for the identification and quantification of one or more biomolecules in a sample containing a mixture of substances, wherein said method comprises the steps of, a) providing a sample which contains one or more biomolecules, b) providing a reagent for the analysis of biomolecules according to Formula I;

A-Y-PRG Formula I

wherein, A may be present or absent and when A is present, A is H or constitutes at least one functional group for the reversible, covalent or non-covalent binding to a support material, whereby Y is a group comprising at least one chelate function for metals, preferable low in isotopes, and whereby PRG is a reactive group for the selective binding of biomolecules to be analyzed, c) coupling the one or more biomolecules from step a) to the reagent of step b), d) selecting the one or more biomolecules labeled in step c) under the employment of a functional group for the reversible, covalent or non-covalent binding to a support material and removal of the one or more unbound biomolecules, wherein (d) optionally comprises releasing the bound biomolecules from the support material and the elution from the matrix; and f) detecting and identifying the one or more labeled biomolecules by means of Inductively Coupled Plasma-Mass Spectrometry (IC PMS), wherein f) optionally comprises cleaving the one or more biomolecules in said sample wherein, said cleaving may be performed (i) before coupling of the reagent PRG or (ii) after coupling.

In a preferred embodiment of the method according to the invention, prior to the detecting step f), a fractionation step is included and the means of fractionation is selected from the group of (i) fractionation based on chemical properties of the sample and/or the one or more labeled biomolecules, (ii) physical properties of the sample and/or the one or more labeled biomolecules, (iii) both, chemical and physical properties of the sample and/or the one or more labeled biomolecules.

Further the means of fractionation is selected from the group of HPLC, LC, FPLC, spin column separation, affinity chromatography, size exclusion chromatography, hydrophobic interaction chromatography, ion exchange chromatography, chromatofocussing, immuno precipitation, gel based methods, 2DGE, electrophoresis, breakable PAGE gels, isoelectric focussing, free-flow electrophoresis, capillary electrophoresis and separation by centrifugation or ultracentrifugation.

Further, the fractions are further analyzed by additional chemical and biological methods, fractionation, chemical or enzymatical cleavage, fractionation and mass spectrometric methods for quantification and identification of biomolecules present in the fractions.

The arrangement of the groups A, X and PRG may be interchanged.

The one or more biomolecules in the sample are selected from the group of, one or more known biomolecules and wherein the reagent according to Formula I is bound to said one or more known biomolecules, one or more unknown biomolecules and wherein the reagent according to Formula I is bound to said one or more unknown biomolecules.

The known and unknown biomolecule is selected from the group comprising a pyrogen, a pharmaceutical compound, a pharmaceutical lead compound, an allergen, an autoimmunogen, a toxin, a polyclonal antibody, an monoclonal antibody, an antigen, lipid, carbohydrate, peptide, protein, protein complex, amino acid, fatty acid, nucleotide DNA, RNA, PNA, siRNA and microRNA.

The one or more biomolecules present are quantified with the aid of an internal standard wherein the internal standard is a solution containing known metal concentrations of at least one or more metals and/or the internal standard consists of one or more biomolecules of known concentration which are labelled by one or more reagents according to the general Formula I:

A-Y-PRG Formula I

whereby, A may be present or absent and when A is present, A is H or constitutes at least one functional group for the reversible, covalent or non-covalent binding to a support material, whereby Y is a group comprising at least one chelate function for metals, preferable low in isotopes, and whereby PRG is a reactive group for the selective binding of biomolecules to be analyzed;

The one or more biomolecules are present in the sample at an amount of less than 1 millimol, or less than 1 micromol or less than 1 nanomol, or less than 1 picomol, or less than 1 femtomol, or less than 1 attomol or at an amount less than 1 milligram, or less than 1 microgram or less than 1 nanogram, or less than 1 picogram, or less than 1 femtogram, or less than 1 attogram.

The dynamic range of quantities measured in the reaction is higher than 10⁶, higher than 10⁷, higher than 10⁸, higher than 10⁹, higher than 10¹⁰, or higher than 10¹¹.

The dynamic range of the quantities of biomolecules analyzed can vary between 10⁰ and 10⁹ in concentration and if two or more biomolecules are analyzed their respective concentrations may vary between 10⁰ and 10⁹ in concentration.

The method according to the invention may be used for the detection of peptides in a biological sample and/or for determining the relative and/or absolute expression of proteins in a protein-containing sample.

The method according to the invention for performing quality control assays during or after production processes in the field of chemistry, food production, pharmacy, production of recombinant proteins and peptides, amino acids, carbohydrates, lipids, fatty acids and diagnostic assays.

The described method for the identification and quantification of one or more biomolecules in a sample containing a mixture of substances allows the sensitive quantification of biomolecules by labeling the biomolecules with metal chelate reagents of the Formula A-Y-PRG. Whereby, A may be present or absent and when A is present A is H or constitutes at least one functional group for the reversible, covalent or non-covalent binding to a support material, whereby Y is a group comprising at least one chelate function for metals being preferable low in isotopes, and whereby PRG is a reactive group for the selective binding of biomolecules to be analyzed. Group A allows a selective enrichment of the labeled biomolecules by antibodies using its hapten properties. Or group A consists of a chemical or biochemical group like biotin which can be used for affinity purification using specific support materials. In the case of biotin the support material comprises e.g. of streptavidin on its surface.

The labeled and to be analyzed biomolecules are detected and identified by mass spectrometric and/or fluorescence methods like Inductively Coupled Plasma-Mass Spectrometry, Laser Induced Fluorescence Transient (LIFT) detection and/or other mass spectrometric and/or fluorescence detection methods (MALDI-MS, ESI-MS, ESI-MSMS). In the case of Inductively Coupled Plasma-Mass Spectrometry liquid samples containing the labeled biomolecules are analyzed for example by FIA-ICP-MS or LC-ICP-MS. If the biomolecules are separated by electrophoresis like PAGE or 2D electrophoresis before ICP-MS analyses the electrophoresis gels have to be breakable. This means that gel bands or gel spots containing labeled biomolecules are preferable liquefied before ICP-MS to ensure a reproducible and high sensitive analysis and quantification of the labeled biomolecules by analysis of the whole or a portion of liquefied sample (gel band). Since all organic components are destroyed during ICP-MS analysis only the metals of the metal chelate reagents are detected. Especially by using lanthanides a superior signal to noise ratio and an extreme high sensitivity is ensured by ICP-MS analysis. Thus the describe method allows a very sensitive detection and quantification of biomolecules which is several orders of magnitude more sensitive than MALDI-MS and ESI-MS methods. Furthermore the dynamic range of quantities measured by ICP-MS is higher than 10⁶, higher than 10⁷, higher than 10⁸, higher than 10⁹, higher than 10¹⁰, or even higher than 10¹¹ and surpasses the dynamic range of quantities measured by MALDI-MS or ESI-MS methods in at least two to six orders of magnitude. This huge dynamic range allows for the first time the accurate quantification of biomolecules which are present in highly different amounts in a sample. The dynamic range of protein amounts or protein concentrations in tissues, cell cultures, body fluids, microorganisms and other biological samples is higher than 10⁶-10⁸. For example it is well know that proteins like transcription factors are present in only a few copies within a cell whereas structural proteins are present in millions of copies, e.g. in plasma albumin makes up about 60% of the total protein concentration and is, thus, the most abundant protein whereas biomarkers like tumor markers are mostly present at very low concentrations (<0.01%). Also in many other fields like chemistry, food production, pharmacy, production of recombinant proteins and peptides, amino acids, carbohydrates, lipids and fatty acids and diagnostic assays it is often a problem to ensure a reliable identification and quantification of biomolecules or chemical molecules especially if these biomolecules or molecules are present at very different amount in a sample containing a mixture of substances. For example in pharmacy often highly effective (protein) drugs are mixed together with other proteins like human albumin to ensure stability and tolerance of the drug and to reduce adsorption to surfaces which could result in lost of drug's biological effectivity and compatibility. These other proteins are often added in much higher concentrations than the drug. In the case of highly effective toxins like botulinum toxin the amount of the drug is in the range of picograms to nanograms whereas in many hundred micrograms or milligrams of human albumin are added. Today MALDI-MS and ESI-MS used for bioanalytics can not cover this high dynamic range of biomolecule concentration and therefore a reliable detection and quantification of biomarkers and drug targets is often unrivaled. Furthermore, the sensitivity of ICP-MS and LIFT is unrivaled. ICP-MS can be easily calibrated for absolute quantification by using several metal standard solutions of different concentrations.

The method described herein, allows the quantification of biomolecules with the aid of an internal standard. The internal standard is a solution containing known metal concentrations of at least one or more metals and/or the internal standard consists of one or more biomolecules of known concentration which are labeled by one or more reagents according to the invention. In the case of proteome analysis the internal standard can be a protein or proteome sample of the same source like the samples to be analyzed. The internal stand is labeled by a different metal than the samples. The internal standard sample is added to each analysis and thus allows a reliable comparison, quantification and normalization of the analyses of all samples to be compared. In IC PMS the different metals used for metal labeling of the samples are quantified.

The describe method is especially appropriate for quality control assays during or after production processes in the field of chemistry, food production, pharmacy, production of recombinant proteins and peptides, amino acids, carbohydrates, lipids and fatty acids and diagnostic assays. It allows identification and quantification of one or more known and unknown biomolecules selected from the group comprising a pyrogen, a pharmaceutical compound, a pharmaceutical lead compound, an allergen, an autoimmunogen, a toxin, a polyclonal antibody, an monoclonal antibody, an antigen, lipid, carbohydrate, peptide, protein, protein complex, amino acid, fatty acid, nucleotide DNA, RNA, PNA, siRNA, microRNA.

In the case of pharmaceutical compounds protein and peptide drugs like hormones, peptide hormones, cytokines, insulin, interleukin-2, somatotropin, glucagons, erythropoietin beta, interferon gamma, Interferon alfa, erythropoietin alpha, GM-CSF, interferon alpha-2a, Faktor VIII, G-CSF, plasminogene-activator, glucocerebrosidase, humane DNAse, follitropine alpha, Interferon beta, Faktor VII, follitropin beta, plasminogene-activator r-PA, Faktor IX, Interferon beta hirudine, desirudine, calcitonin, aprotinin, alfacon-1, growth factor r-hPDGF, botulinum toxin, AB-toxins (botox toxin, cholera toxin, vero toxin, shiga toxin, diphteria toxin, pertussis toxin), Hepatitis-A/B/C/D/E (HBs-antigen: Hepatitis B-surface-antigen, Australia-antigen, Anti-HBs: Hepatitis B-surface-antibody, Anti-HBc: Hepatitis B-Core-antibodies, Anti-HBc-IgM, Hepatitis B-Core-IgM-antibodies, HBe-Antigen, Hepatitis Be-antibodies, HA-antigen, Hepatitis A antigen, anti-HAV, Hepatitis A-antibody, anti-HAV-IgM: Hepatitis A-IgM-antibody, Anti-HCV, Hepatitis C-antibody, proteases, nucleases, Ab-toxines, Hep ABCDE or simply any monoclonal antibody can be accurately quantified.

For identification, characterization and relative quantification of biomolecules the describe method uses MALDI-MS and ESI-MS in addition to quantification by ICP-MS. For example a fractionation step is included before ICP-MS which allows a further analysis of biomolecules. In the case of protein analysis or proteome analysis the fractions are further analyzed by additional chemical and biological methods, protein fractionation, chemical or enzymatical cleavage, fractionation of peptides and mass spectrometric methods for quantification and identification of biomolecules present in the fractions. This allows the identification and quantification of regulated and non-regulated proteins, drug targets and biomarkers within the sample's fraction to be analyzed.

The describe method for the identification and quantification of one or more biomolecules in a sample often requires fractionation methods for separating the biomolecules to by analyzed before MS and LIFT analysis. All know separation methods for biomolecules can be used for this purpose like HPLC, LC, FPLC, spin column separation, affinity chromatography, size exclusion chromatography, hydrophobic interaction chromatography, ion exchange chromatography, chromatofocussing, immuno precipitation, gel based methods, 2DGE, electrophoresis, breakable PAGE gels, isoelectric focussing, free-flow electrophoresis, capillary electrophoresis and separation by centrifugation or ultracentrifugation.

Approach for Quality and Product Control

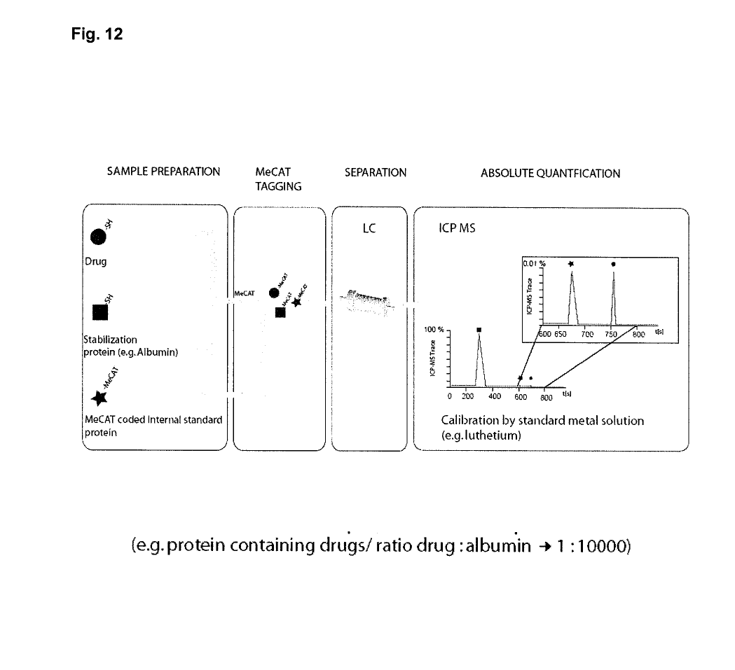

A protein drug containing a drug to albumin ration of 1:10000 is analyzed by metal labelling according to the invention. An internal standard protein of known concentration and labelled with a different metal is added. The sample is separated by LC or other separation methods and applied to ICP-MS for absolute quantification. Due to the outstanding sensitivity and dynamic range of quantities measured by ICP-MS the different components are accurately quantified. External standard metal solutions are used for calibration (see FIG. 12).

Approach for Proteome Analysis

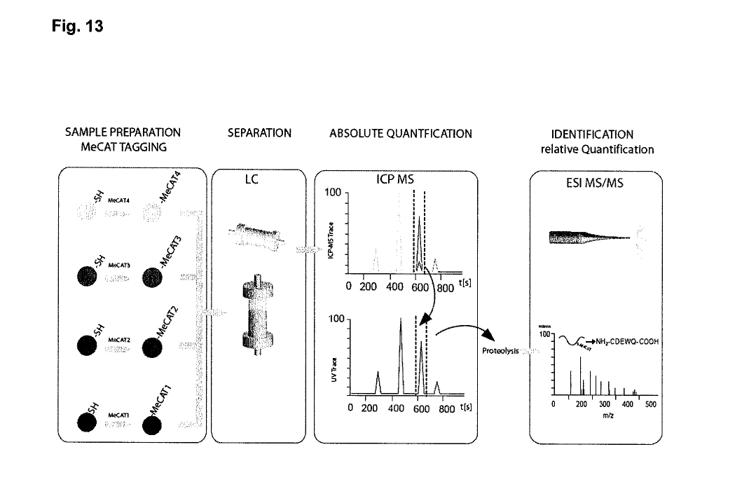

The metal labelled proteins of the different samples are combined and fractionated by LC or other separation methods. ICP-MS is used for quantification of the separated samples. Fractions of interest (e.g. fractions with altered concentrations of the samples) are fractionated. The proteins are separated again and/or chemically and/or enzymatically hydrolyzed in peptides and identified and quantified by LC-MS (FIG. 13).

Approach for Pharmacokinetic Quantification

A protein drug or peptide drug from a complex sample containing various biomolecules is analyzed by metal labelling according to the invention. Complex sample means a sample containing at least two or more proteins or peptides. In a preferred embodiment complex sample means a sample like tissue, cell or body fluid. In a first step the biomolecules in the sample are preferentially all labelled with one particular label. In a preferred embodiment that means all proteins and peptides. Additionally biomolecules in additional samples are labelled with independent labels allowing multiplex analysis of several samples. In a second step a purified sample of the drug or purified samples of biomolecules which should be quantified are labelled with one or more independent labels. They are used as internal standards for quantification of the corresponding biomolecule or biomolecules in the complex sample or samples. The differentially labelled protein samples from step 1 and step 2 are mixed. The sample is separated by LC or other separation methods and applied to ICP-MS for absolute quantification. Due to the outstanding sensitivity and dynamic range of quantities measured by ICP-MS and/or LIFT the different components are accurately quantified. The purified sample of the drug labeled with an independent label (from step 2) comigrates with the corresponding protein or peptide of the sample or samples labeled in the first step. External standard metal solutions are used for calibration (see FIG. 12).

The reagent for the mass spectroscopy analysis of peptides has the general formula:

A-Y-PRG

wherein, A constitutes at least one functional group for the reversible, covalent or non-covalent binding to a support material, in which Y is a group comprising at least one chelate function for metals being low in isotopes, and in which PRG is a reactive group for the selective binding of peptides or other biomolecules to be analyzed.

In an alternative reagent according to the present invention, the arrangement of the groups A, X and PRG may be interchanged. Indeed, the reagent according to the invention may be present with its components being arranged in different ways, so far as all functional requirements for the performance of MeCAT are still met.

Preferably, the function PRG is selected from a sulfhydryl-reactive group, an amine-reactive group and an enzyme substrate. It is moreover preferred, that PRG is selected from the group of an amine-reactive pentafluorophenyl ester group, an amine-reactive N-hydroxysuccinimide ester group, sulfonylhalide, isocyanate, isothiocyanate, active ester, tetrafluorophenyl ester, an acid halide and an acid anhydride, a homoserine lactone-reactive primary amine group and a carboxylic acid-reactive amine, alcohol or 2,3,5,6-tetrafluorophenyltrifluoro-acetate, a iodine acetylamide group, an epoxide, an α-haloacyl group, a nitrile, a sulfonated alkyl, an arylthiol and a maleimide.

Particularly preferred is a reagent according to the invention, in which A is selected from biotin or modified biotin, a 1,2-diol, glutathione, maltose, a nitrilotriacetic acid group, an oligohistidine or a hapten. In case of biotin, the reagent can e.g. be coupled to a streptavidin group in order to allow its convenient isolation. Particularly preferred in this context is the employment of a streptavidin-labeled column matrix or of coated beads.

In a further embodiment, A is a reactive group coupled to the support material, which reactive group can again be cleaved off the support material. Possible options for this are—among other things—disulfide bonds (S—S), which can be reduced again, thus leading to cleavage, or photosensitive bonds, which can be cleaved by exposure to light.

In a further embodiment of an inventive reagent according to the present invention, the reagent includes a chemically and/or enzymatically cleavable linker between the groups A, X and/or PRG. In general, this linker can be made up of CH₂-groups, which are located between the groups A, X and/or PRG, thereby joining these groups. One or more of the CH₂-groups can be substituted, wherein the character of the substitutions is not relevant, so far as the functions of the groups A, Y and PRG are not affected. Advantageously however, one can introduce further functions via the linkers, like e.g. the chemical and/or enzymatic cleavability mentioned above. Possible substitutions are alkyl, alkenyl and alkoxy groups, aryl groups, which may be substituted with one or more alkyl, alkenyl, alkoxy and aryl groups, acidic groups and basic groups. Moreover, double and triple bonds may be present within the linker, and heteroatoms like e.g. O, S and N may be inserted, e.g. in the form of a linker containing a disulfide group.

An essential function of the reagent according to the present invention is its chelate forming function. In preferred reagents according to the present invention, Y is selected from a macrocyclic lanthanoid chelate complex, a functionalized tetraaza-macrocycle, a polyaza-polyacetic acid, DOTA, a DOTA-derivative, NOTA, a NOTA-derivative, EDTA, DTPA-BP, DTPA, DO3A, HP-DO3A and DTPA-BMA. Particularly preferred compounds are 1,4,7,10,13,16,19,22-octaazacyclotetracosane-1,4,7,10,13,16,19,22-octaacetic acid (OTEC), and 1,4,7,10,14-17,20,23-octaazacyclohexacosane-1,4,7,10,14,17,20,23-octaacetic acid (OHEC).

The metals, which can be bound by the chelate-forming function of the reagent according to the present invention, can be selected from a large variety of metals, thereby significantly improving the flexibility when using the reagent according to the present invention. Thus, the metal bound by the chelate complex can be selected from Ag, Al, As, Au, Be, Cd, Ce, Co, Cr, Cu, Dy, Er, Eu, Fe, Gd, Hg, Ho, In, La, Li, Lu, Mn, Na, Nd, Ni, Pb, Pr, Rb, Rd, Sb, Sm, Sn, Tb, Tl, Tm, V, W, Y, Yb and Zn. According to the invention, the chelate-forming group can be labeled with several different metals.

A further aspect of the present invention relates to the use of the reagent according to the invention for the detection of peptides in a biological sample and/or the determination of the relative expression of proteins in a protein-containing sample. In this context, the biological sample can be a sample taken directly or a pre-fractionated sample for the differential investigation of the proteome of one, two or more cell, tissue or bodily fluid samples. Also investigated however can be other protein-containing samples, like e.g. protein fractions from organelles or other cellular compartments. The method is preferably applied in the course of diagnosing or monitoring the disease of an animal, in particular the human, by detecting the relative expression of proteins in a protein-containing sample taken from the animal. By the analysis and elucidation of differentially expressed proteins, one can draw conclusions about proteins being involved in diseases on a cellular level, which proteins may serve as targets for therapeutic substances or may be useful for the diagnosis and monitoring of a therapy.

A further aspect of the present invention relates to an analysis set (kit) for diagnosis, containing at least one reagent according to the present invention together with further substances and/or enzymes suitable for the detection of peptides in a biological sample and/or the determination of the relative expression of proteins in a protein-containing sample; in particular containing an internal standard. By means of this kit, one can e.g. perform a proteome labeling, the products of which can then be sent to a central analytical laboratory for analysis by mass spectrometry.

In a further variant of the method according to the invention, one may consider the employment of radioactive metal ions, which allows for a particularly sensitive detection, e.g. by scintillation counting. The respective ions are very familiar to the expert in the field of radiochemistry and may be gathered from any common chemistry textbook, such as for example the Römpp-Lexikon Chemie, 10^{th} edition, Thieme Verlag, Stuttgart.

From the view of a chemist, the entirety of possibilities for a rapid quantitative protein analysis or analysis of protein functions is by far not exhausted by the ICAT-method. The basic idea of the class of reagents presented herein is the clever combination of three different functions in one molecule; i) the possibility to specifically bind to a protein, ii) the possibility to easily separate labeled peptides from unlabelled peptides after enzymatic or chemical hydrolysis, and iii) the possibility to relatively quantify peptide pairs derived from different samples (e.g. from cells of a species in different developmental phases) via the mass difference of corresponding peptides in the mass spectrum.

The first two functions are employed in many common analytical separation methods. The third function is associated with the most modern MS-technique in combination with the newest computer-aided database search programs, which allow for the identification of a protein in dependence on the amino acid sequence of one single peptide or a few peptides (e.g. cysteine-containing peptides).

The advantages of this method are obvious: Each available amount of starting material can be processed. Also proteins only present in minimal amounts are detectable and quantifiable, since they are enriched by means of a cysteine-specific selection. By means of other amino acid specific or substrate specific functional groups in the MeCAT reagent, further proteins can be reliably determined in the analysis. The complexity of the peptide mixture is reduced this way, thus allowing for a largely reduced expenditure of work and a more rapid and successful protein identification via data base search programs.

Instead of an isotope label, the present invention provides the integration of a metal chelate complex into the reagent as an alternative. A concept, how these reagents may be designed, is illustrated in FIG. 1.

The synthesis of an isotope-labeled linker is very expensive and not always possible, since, as it is generally known, there only is a very limited number of stable isotope reagents such as ²H, ¹³C or ¹⁵N. This e.g. means that samples derived from a very limited number of cell cultures, which have been exposed to different conditions, can be investigated and compared in respect to the quantitative and qualitative detection of dynamic changes in protein production. Literature describes examples for the comparison between two samples with ¹H- and ²H-labelled ICATs.

Metal ions are available in a much greater variety and at lower price. It just depends on finding suitable ones and packing them appropriately into the amino acid specific reagent thereby preventing them from getting lost by dissociation or exchange reactions.

The candidate chelate ligand must stabilize the metal ion well enough that the complex remains completely intact during the entire process, that its stability is ensured also in case of larger pH changes, and that no exchange processes with the peptides as potential ligands can happen. The solubility characteristics of the complex are not allowed to largely differ from those of the other components of the reagent, i.e. the protein-reactive functional group and the molecule portion for chromatographic separation purposes. The entire molecule preferably has to be soluble in the sample solution in order to ensure an efficient interaction of the tag with the specific protein binding sites.

For quick and unambiguous protein identification, one can integrate into the protein-reactive reagent a metal ion, which normally is not found in proteins and which has a very characteristic isotope pattern. Such a metal ion will be very easily detected in the mass spectrum of the labeled peptide. Computer algorithms can automatically compare the experimentally observed isotope pattern of the mass fragment, with or without the metal ion or mass specific labeling. This causes no greater demand on the sensitivity of the employed mass spectrometer. In contrast, the complexing agents bound to the peptides will positively affect the sensitivity of detection, since complex forming agents are known as strongly contaminating substances in the mass spectrometry of peptides, thus normally requiring avoiding them even at the lowest concentrations. Via the automatic screening of the mass spectra of all peptides separated by 2DLC or another suitable method, it should be possible to very rapidly and unambiguously select the cysteine-containing peptides for reason of their isotope pattern from a peptide mixture mainly containing peptides without cytosine residues. Only the exactly determined mass of these selected peptides is used for protein identification by correlating the experimental data with the data from genome and protein databases. The sequencing of peptides by CID-MS allows for the identification.

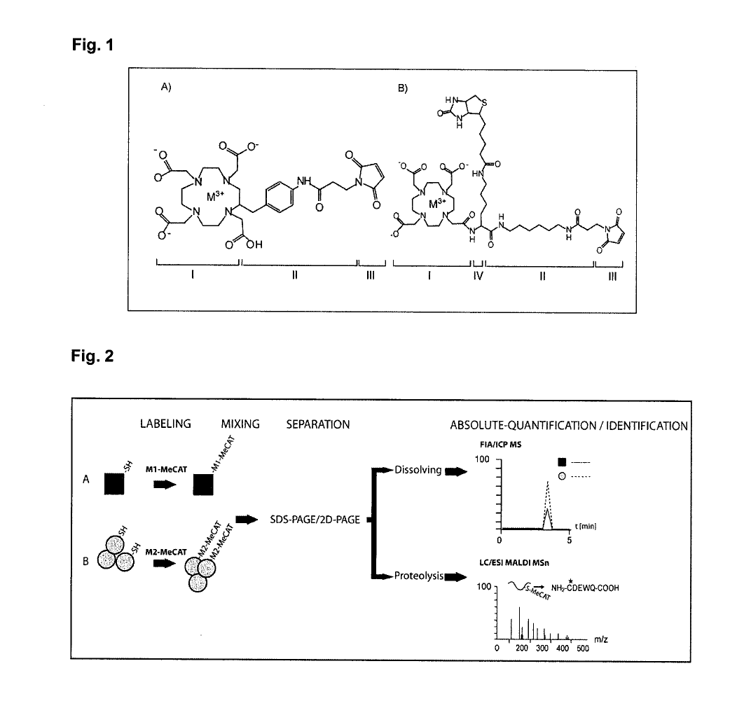

For the relative protein quantification and characterization in several protein samples, several metal chelate complexes with identical ligand portion, but with different metal ions come into question, wherein these complexes have to be such resembling in respect to their thermodynamic stability and their kinetic behavior that metal exchange processes between them can be ruled out. The relative atomic masses of the metal ions should not differ by more than 10 Daltons in order to detect matching peptide pairs easily in the mass spectrum. The metal ions moreover should be low in isotopes in order to avoid unnecessary complication of the assignment. Besides the protein-reactive functional group, the metal ion specific reagent may comprise a molecular component for separating the labeled peptides after protein hydrolysis by means of column chromatography. FIG. 2 schematically illustrates the preferred strategy for quantifying the protein expression by means of metal specifically labeled reagents (MeCATs/MeCODs).

For the efficient binding of the metal ion, macrocyclic ligands are particularly suitable as chelates, since they are characterized by a high thermodynamic stability and a kinetically inert behavior in respect to dissociation. For reason of their topological characteristics, the macrocycles provide a multiplicity of strategically distributed donor atoms, which, in case of a suitable conformation and dimension of the ligand can interact in an effective manner with the metal ion. A “statistical stabilization” follows from the very low probability of a simultaneous break up of all metal-ligand-donor-bonds. Similar to the receptor binding sites of enzymes, a large number of coordinate interactions, which are only weak as single interactions, lead to a binding of the metal ion, which, in case of a suitable molecular architecture, is not just stable, but also selective. Thereby, in contrast to ligands with open ligand chains, the exchange with biologically relevant metal ions is effectively prevented.

The present invention relates to a method and a reagent suitable for performing said method, which method includes a reproducible, systematic, qualitative and quantitative proteome characterization by means of non-isotope metal coded markers and—among other items—the most modern tandem methods of mass spectrometry.

The metal code is a macrocyclic lanthanoid chelate complex on the basis of DOTA (1,4,7,10-tetraazacyclododecane-1,4,7,10-tetraacetic acid) or a transition metal complex on the basis of NOTA (1,4,7-triazacyclononane-1,4,7-triacetic acid), which has to be equipped with an amino acid specific functional group and a further molecular component for the chromatographic separation of the labeled peptides. The marker to be synthesized must display a good solubility in water and a high kinetic stability. Compounds with different lanthanoid ions must not significantly differ in respect to their chemical reactivity and physical properties. The metal coded markers are characterized in respect to their structure, their thermodynamic properties in aqueous solution and their reactive behavior towards peptides. Their reproducible application in proteome analysis has to be tested in model experiments and real samples in combination with multidimensional chromatography, MS/MS and database analysis.

The metal coded markers are covalently bound to the proteins of cell lysates in a “site-specific” manner. After the proteolysis of the proteins, the metal labeled peptides are isolated chromatographically and are further fractionated in order to be then quantified by mass spectrometry and, in the second step, to be sequenced. By means of a direct quantitative comparison of well determined states, one features differences in the protein composition, which then have to be correlated with biological effects.

In combination with a data base search, it is possible to identify the basal proteins of interest via one single or just a few peptides.

In this field of coordination chemistry, there exist lots of works from the last 15-20 years, the disclosure of which can be readily resorted to in the context of the present invention.

The cyclic ligand, which preferably may be a functionalized tetraaza-macrocycle, i.e. a derivative of DOTA (1,4,7,10-tetraazacyclododecane-1,4,7,10-tetraacetic acid) or a triaza-macrocycle, a derivative of NOTA (1,4,7-triazacyclononane-1,4,7-triacetic acid), is either constructed of amino acids, or synthesized according to a very efficient, template-assisted cyclization reactions, which has been recently developed in the applicants' work group. The protein-reactive group and the function for peptide isolation (e.g. a specific amino acid for the covalent binding to a column containing isothiocyanate groups or biotin for affinity chromatography) can be integrated into the carbon scaffold of the macrocyclic ligand, or the metal chelate complex is suitably connected with the protein-reactive group and the function for peptide isolation via a linker.

Suitable as metal ions for the NOTA-ligand are transition metal ions like copper(II), nickel(II) and zinc(II).

Suitable metal ions for the DOTA-like ligands are the lanthanoid ions, which will form very stable complexes with comparably high complex stability constants and very similar molecular weights with this class of ligands. They are very similar in respect to their chemical properties, and the contraction of the ion radius in consequence of the mass increase in case of the very well studied lanthanoid-DOTA-complexes (DOTA=1,4,7,10-tetraazacyclododecane-1,4,7,10-tetraacetic acid) only has a minute influence on the kinetic stability of the lanthanoid chelate complexes. The high in vivo stability of these compounds led to the successful employment of the DOTA-gadolinium(III)-complex as a contrasting agent in magnetic resonance tomography. For in vivo applications, the kinetic stability of the complex is much more important than their thermodynamic stability. An inert complex will not show any ligand or metal exchange reactions, even when the thermodynamic stability constant is not very high. One reason for the DOTA-lanthanoid-complexes being both very stable and inert, is the optimal size relation between the metal ion and the cavity provided by the ligand. Metal ion and ligand constitute a fixed, well locked structure, which under physiological conditions shows an extremely slow dissociation and can only be attacked by protons when being in an acidic medium. [Gd(DOTA)(H₂O)]⁻ shows a half-life of 200 days in an aqueous solution at pH 5 and a half-life of 85 days at pH 2. The well investigated metal exchange reaction between [Gd(DOTA)]⁻ and [Eu(DOTA)]⁻ in the pH-range 3.2-5.0 shows, that the velocity determining step of this exchange reaction is the proton-assisted dissociation of [Gd(DOTA)]⁻. Even when a protonation takes place at the acetate groups, these mono- and di-protonated complexes are not reactive, since the metal ion is located within the coordination cage. To achieve destruction of this cage, the protons have to be transferred to the N-atoms. This process only takes place extremely slowly via a rearrangement of the entire complex. On the basis of this investigation, metal exchange reactions between DOTA-lanthanoid complexes in the relevant time interval and pH range can be excluded with a very high probability.

### EXAMPLES

The novel strategy presented here allows exactly this, the absolute quantification of proteins and peptides. The method uses chemical labeling and affinity chromatography when necessary, but instead of isotopes, the lanthanides in macrocylic complexes are employed. Using electrospray, structures can be determined, but the quantitative information comes from measurements with inductively coupled plasma mass spectrometry (ICP MS) (Beauchemin, D., Kisilevsky, R. (1998) A method based on ICP MS for the analysis of Alzheimer's amyloid plaques. Anal Chem 70,1026-9).

An accurate and sensitive quantification of elements out of almost every biological sample is amenable and makes this ionization technique suitable for quantification of metal-labeled low abundant proteins in combination with enrichment and depletion procedures. The technique provides extreme detection capability and it is unaffected by chemical structures (Siethoff, C., Feldmann, I., Jakubowski, N., Linscheid, M. (1999) Quantitative determination of DNA adducts using liquid chromatography/electrospray ionization mass spectrometry and liquid chromatography/high-resolution inductively coupled plasma mass spectrometry. J Mass Spectrom 34, 421-6.), which allows the present invention to use a simple internal standard containing an appropriate metal. 80 pg/g (ppt) for 157Gd and 2 fg/g (ppq) for 175 Lu have been reported by Zhang et al. (Zhang, A., Liu, X., Zhang, W. (2004) Determination of rare earth impurities in high purity europium oxide by inductively coupled plasma-mass spectrometry and evaluation of concentration values for europium oxide standard material. Eur J Mass Spectrom (Chichester, Eng) 10, 589-98.) and Thermo Electron Corporation as limits of detection. The molecular structures of the metal labels used are shown in FIG. 1.

Reagents and Workflow

We used two different labelling reagents, i.e. MeCATBnz and MeCATBio (FIG. 1). They contain a maleimide as the thiol specific group and the DOTA macrocycle, which has been used previously as an immunoglobulin recognition site for affinity purification (Whetstone, P. A., Butlin, N. G., Corneillie, T. M., Meares, C. F. (2004) Element-coded affinity tags for peptides and proteins. Bioconjug Chem 15, 3-6). The two reagents differ in the spacer region, which for MeCATBnz is short, whereas MeCATBio in addition possesses a biotin bearing spacer to allow affinity purification. The reaction of the DOTA macrocycle with lanthanide metal ions (M3+) results in very stable metal-chelat-complexes (see table 1)

TABLE 1

| | ¹⁵⁹Tb | ¹⁶⁵Ho | ¹⁶⁹Tm | ¹⁷⁵Lu |
|---|---|---|---|---|
| Exact Mass [g mol⁻¹] | 158.92535 | 164.93032 | 168.93421 | 174.96700 |
| Complexing Constants | 24.22 | 24.54 | 24.41 | 25.41 |
| DOTA [logK] | | | | |

The different masses allow distinguishing differently labeled species using ESI-MS and IC PMS.

For the quantitative analysis of a protein mixture, MeCAT was combined with high-resolution 2-D electrophoresis using dissolvable gels (DPAGE, Proteome Factory AG, Germany). Samples were denatured and reduced before reaction at the cystein residues with M-MeCAT reagents (M=Lanthanides). The samples were mixed together and divided into two portions, before subjected to an analytical and micropreparative 2-D electrophoresis, respectively. After staining the spots of interest they were dissolved for quantification by FIA/ICP MS or digested for identification using HPLC/MSⁿ. The current workflow is displayed in FIG. 2.

Labeling of Peptides

The commercially available peptides (Bachem, Weil am Rhein, Germany) were reduced with 2 mM TCEP at 37° C. for 30 min, before adding a tenfold excess of M-MeCAT_{Bio} reagent (M=Lu, Ho, Tb, Tm). The modification was carried out in 10 mM Hepes (pH 6.5) and left for 2 h at 37° C. Finally, the reaction was stopped using an excess of DTT.

Labeling of Standard Proteins

The standard proteins used for this study were Bovine Serum Albumin (Bos taurus) and α-Lactalbumin (Bos taurus) from SIGMA Aldrich. 1.25 nmol of protein were reduced in 2 mM TCEP for 30 min at 37° C. Reduced proteins were labeled using different M-MeCAT_{Bnz} reagents (M=Lu, Ho, Tm) in a twenty fold excess to each cysteine residue. The reaction was carried out in a solution containing 5 mM EDTA, 50 mM NaOAc, 5% acetone for α-lactalbumin and 5 mM EDTA, 25 mM NaOAc and 10% acetone for BSA. Acetone and EDTA were used to achieve complete denaturation of proteins. The reaction mixture was left for 12 h at 37° C. and stopped using DTT.

Labeling of Proteins for 2-D Electrophoresis

Fresh eyes from one year old Sus scrofa were obtained from the slaughter house and the lenses were immediately prepared on ice as described (Dewey, J., Bartling, C., Rae, J. L. (1995) A non-enzymatic method for lens decapsulation which leaves the epithelial cells attached to the fibers. Curr Eye Res 14, 357-62.). The prepared Sus scrofa lens proteins were dissolved in 9 M Urea, 70 mM TCEP and 1% CHAPS. The amount of protein was determined by Bradford assay. 250 μg protein of lens sample A and B were reduced with 10 mM TCEP for 30 min at room temperature. Reduced proteins (sample A, B) were labeled with MeCAT_{BNZ} (Lu, Tm) each with 800 μg. The reaction was carried out in a solution containing 5 mM EDTA, 50 mM NaOAc, 10% acetone and was left for 12 h at 37° C. The reaction was stopped using excess of DTT.

Affinity Purification

Prior to affinity purification, 35 μl of water and 10 μl of tenfold binding buffer (500 mM Tris-HCl, pH 7.4 and 10 mM DTT) were added to 55 μl solution containing 20 pmol M-MeCAT_{Bio} (M=Ho, Lu, Tm, Tb) labeled peptide (HIV-integrase inhibitor) and 10 μg of E. coli cell lysate tryptic digest. For affinity purification of MeCATBio labeled peptides, a modified protocol of Girault et al. (Girault, S., Chassaing, G., Blais, J. C., Brunot, A., Bolbach, G. (1996) Coupling of MALDI-TOF mass analysis to the separation of biotinylated peptides by magnetic streptavidin beads. Anal Chem 68, 2122-6.) using streptavidin-coated (SA) magnetic beads (M-280, Invitrogen, Karlsruhe, Germany) was used. After incubation and immobilization of 10 μl, SA magnetic beads with a magnetic concentrator, the supernatant was removed and the beads were washed twice with 100 μl, cleaning buffer (500 mM Tris-HCl, pH 7.4, 10 mM DTT and 1 mg/ml BSA) followed by 100 μl, binding buffer (50 mM Tris-HCl, pH 7.4 and 1 mM DTT). Next, the beads were mixed with 100 μl of the prepared peptide mixture and incubated for 60 min at 25° C. with gently shaking. The supernatant was removed and the beads were washed four times with 100 μl washing buffer (50 mM Tris-HCl, pH 7.4 and 0.01% m/v SDS), four times with 100 μl 1 mM DTT and finally three times with 100 μl water. After removal of the supernatant, peptides were eluted with 50 μl of 0.1% (v/v) TFA, 40% (v/v) ethanol by incubation for 5-10 min at 60° C. and the supernatant containing the eluted labeled peptides was dried in a Speedvac and stored at −20° C. until further analysis.

SDS-PAGE to Monitor Efficiency of Metal Coded Labeling.

To demonstrate labeling efficiency, MeCAT coded proteins were subjected to SDS-PAGE as described by Laemmli using 4% w/v stacking gel and 15% w/v separating gels after labeling reaction. To visualize the mass shift of metal coded proteins, gels were silver stained (Heukeshoven, J., Dernick, R. (1988) Improved silver staining procedure for fast staining in PhastSystem Development Unit. I. Staining of sodium dodecyl sulfate gels. Electrophoresis 9, 28-32) or stained with a colloidal Coomassie G 250 solution (Klose, J., Kobalz, U. (1995) Two-dimensional electrophoresis of proteins: an updated protocol and implications for a functional analysis of the genome. Electrophoresis 16, 1034-59).

2-D Electrophoresis

Without further purification or fractionation the M-MeCAT labeled samples (M=Tm, Lu) were subjected to 2-D electrophoresis analysis. Soluble proteins were separated by the high-resolution 2-D electrophoresis technique according to Klose and Kobalz (see above). Large (30×23 cm) soluble 2-D gels (DPAGE, Proteome Factory AG, Germany) were prepared with ready-made gel. Analytical and preparative gels were 1 mm thick. For analytical runs 100 μg and for preparative runs up to 500 μg of the combined lens samples were applied at the anodic side of the gel. In the first dimension vertical IEF using carrier ampholytes in the range of pH 2-11 was performed. The second dimension was a vertical SDS-PAGE as described by Laemmli using 15% w/v DPAGE gels. After the run, the analytical gels were stained with silver nitrate and preparative gels were stained with CBB G-250. For the following analysis protein spots were either in-gel digested or solubilized, according to the DPAGE protocol for FIA/ICP MS analysis.

Digestion

For in gel digestion the gel pieces were cut out and destained (10% acetic acid, 30% ethanol and 60% water v/v). Then, the pieces were rinsed with 100 μl 50 mM ammonium hydrogen carbonate buffer and dehydrated with 100 μl 100% acetonitrile four times and dried in a Speedvac for 15 min. 50 μl of 50 mM ammonium hydrogen carbonate buffer and 2 ng trypsin were added to each dehydrated gel piece; the digestion was carried out for twelve hours at 37° C. The samples were centrifuged and the supernatant was carefully removed before the gel pieces were incubated in 50 μl of 50 mM ammonium hydrogen carbonate buffer for 30 min, centrifuged and freed from the supernatant. Finally, all pieces were extracted with 50 μl acetonitrile and all supernatants were pooled and analyzed.

Peptide Analysis by Nano-LC/ESI MS/MS

Digested samples were dissolved in 0.1% (v/v) formic acid to a final concentration of 500 fmol/μl. They were analyzed with a Agilent 1100 nano-LC system (Agilent, Santa Clara, US), consisting of an autosampler WPS, nano pump and iso pump. Columns were a Zorbax 300SB-C18 3.5 μM, 150 mm×75 μM and a Zorbax 300SB-C18 3.5 μM, 0.3 mm×5 mm enrichment column (Agilent, Santa Clara, USA). Separation was carried out using a binary mobile phase water/acetonitrile gradient with a maximum flow rate of 0.29 μl/min. The eluents were: A (iso pump) 98.5% deionized water 1% acetonitrile and 0.5% (v/v) formic acid; B (nano pump) consisted of 94.9% deionized water, 5% acetonitrile and 0.1% (v/v) formic acid; C (nano pump) consisted of 99.9% acetonitrile and 0.1% (v/v) formic acid. For peptide separation, the sample was injected via autosampler onto the enrichment column using a 20 μl flow (A).

The separation column was directly coupled to a Bruker Esquire 3000 plus (Bruker Daltonics, Bremen, Germany) via Nano-ESI source using a 10 μm ID PicoTip emitter (New Objectives, Woburn, Mass., US). For MS and MS/MS experiments, following mode and tuning parameters were used: Scan range: 100-3000 m/z, Polarity: positive, Capillary Voltage: 1550 V.

High resolution MS and MS/MS experiments were carried out using a Finnigan LTQ FTMS (Thermo Fisher Scientific, Bremen, Germany), using nano liquid chromatography system (Agilent 1100) under same conditions (described above) MS and MS/MS experiments were recorded with following parameters: Scan range 350-2000 m/z, Polarity positive, Capillary Voltage: 1600 V, Tube Lens Voltage: 10 V.

Peptide MS Analysis by MALDI ToF MS.

Peptides were analyzed using MALDI on a Bruker-Daltonics reflector ToF mass spectrometer Reflex II (Bruker Daltonics, Bremen, Germany), α-HCCA served as matrix. The samples were prepared by the common dried-droplet method. A mixture of angiotensin I, angiotensin II, and substance P was used for external mass calibration. The accuracy of peptide mass measurement was about 10 ppm.

FIA/ICP MS and LC/ICP Quadrupole MS

For analysis of lens protein samples resolubilized from polyacrylamide gels a combination of Famos Ultimate II (LC Packings, Sunnyvale, Calif., USA) and Elan 6000 ICP MS (Perkin Elmer, Mass., US), Plasma Power 1100 W, Nebulizer Gas Flow 1.5 l/min was used. As interface between LC and ICP MS the microconcentric nebulizer MCN-6000 with a membrane desolvation system was used (Cetac, Omaha, Nebr., USA). Standard solutions for external calibration (18 fmol, 36 fmol, 72 fmol, 144 fmol, 288 fmol, 2.88 pmol) were prepared using a multi-element standard mixture (Merck, Darmstadt, Germany) containing Holmium (Ho) and Lutetium (Lu).

FIA/ICP HR Sector Field MS

Analysis of labeled standard proteins was carried out on an Element XR (Thermo Fisher Scientific, Bremen, Germany) using a Plasma Power of 1450 W, Nebulizer Gas Flow of 0.87 l/min, Nebulizer: concentric PFA in combination with a Surveyor HPLC-System (Thermo Fisher Scientific, Bremen Germany) with a flowrate of 200 μl/min. Standard solutions for external calibration (2.5 fmol, 5 fmol, 10 fmol, 100 fmol, 1000 fmol, 5000 fmol, 10000 fmol) were prepared using a multi-element standard mixture containing Holmium (Ho) and Lutetium (Lu).

Results

To demonstrate MeCAT is a viable alternative with important advantages to other labeling techniques, we showed that the labeling of peptides and proteins is complete, reproducible, and robust. The experiments were carried out on peptide level (HIV-integrase inhibitor, tryptic BSA digest), on protein level (Bovine Serum Albumin, α-Lactalbumin) and the cellular level (eye-lens—Sus scrofa).

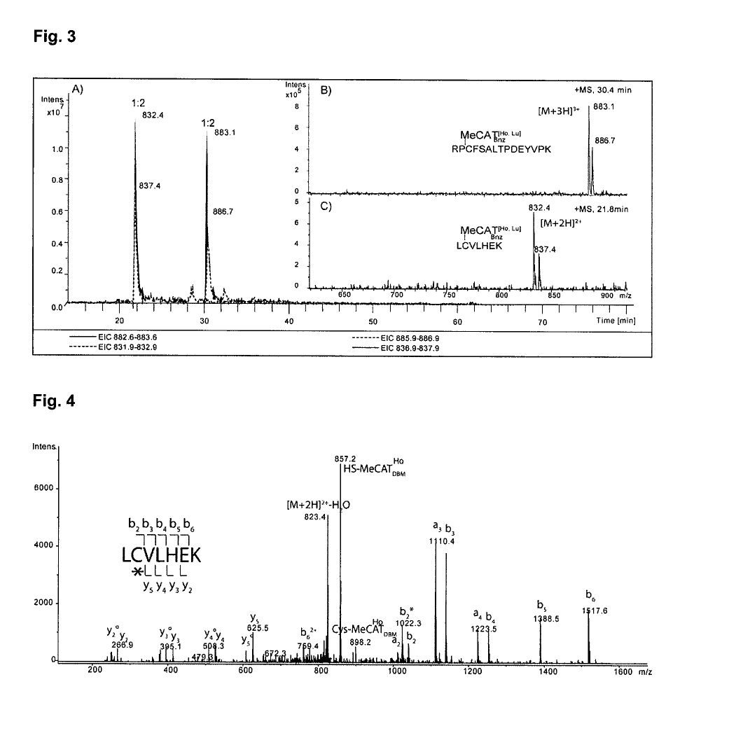

At first, coelution of differently tagged peptides during HPLC was shown using a 2:1 mixture of two tryptic digests of BSA. FIG. 3 shows pairs of peptides labeled with M-MeCAT_{Bnz} (M=Ho, Lu) having the expected ratio of 2:1 (Ho/Lu).

The CID MS² fragmentation pattern of investigated BSA peptides show y- and b-series (FIG. 4). The M-MeCAT_{Bnz} labeled peptides were identified by MASCOT.

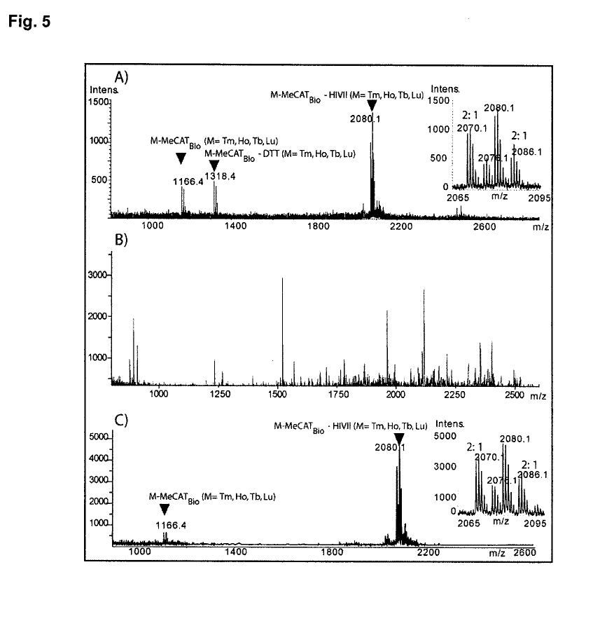

To prove the complete labeling at the peptide level, the HIV-integrase inhibitor (SEQ ID NO:1, HCKFWW) was labeled in four separate reactions with different metal coded M-MeCAT_{Bio} reagents (M=Tb, Ho, Tm, Lu). The resulting reaction mixtures were combined in a ratio of 2:1:2:1 (Tb/Ho/Tm/Lu) for MALDI-ref-ToF MS analysis. A survey mass spectrum of the labeled peptide mixtures is given in FIG. 5A. The expected ratios were found, while no unlabeled peptide was detectable.

Then, the mixture of tagged peptides was spiked in a tryptic digest of an E. coli cell lysate. FIG. 5B shows the survey spectrum of the peptide mixture; only peptides from the E. coli cell lysate are visible. After affinity purification, only the M-MeCAT labeled HIV-integrase inhibitor and traces of M-MeCAT reagents were visible. The ratio of metals remained unchanged (FIG. 5C).

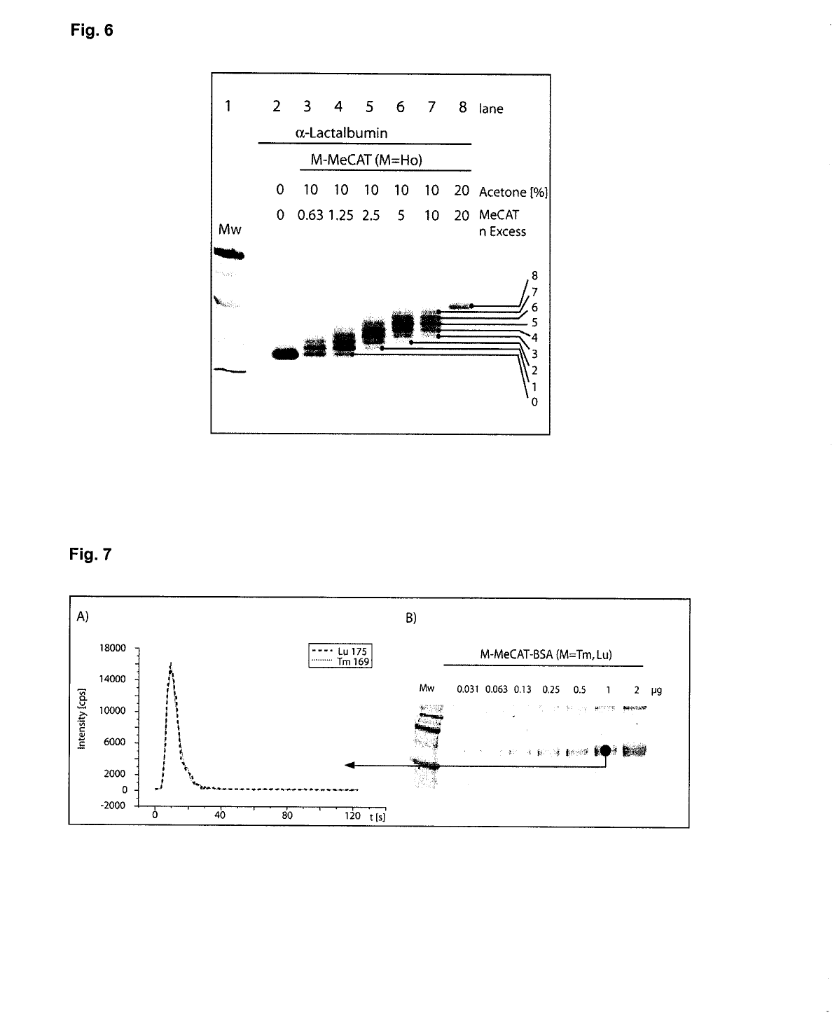

For proteins, the reaction behavior of M-MeCAT_{Bnz} has been tested using BSA and α-Lactalbumin. We monitored the labeling reaction of different ratios of proteins and reagents. For comparison, the products were separated on a 15% SDS-PAGE gel. Because the MeCAT label changes the mass of the proteins and the pl considerably, progress in the reaction should be visible. Thus, the slowest migrating band contains the highest number of tags, whereas the unlabeled protein should be found in the first band. In FIG. 6, the results of the separation of differently tagged α-Lactalbumin is shown; depending on the excess of MeCAT_{Bnz} reagent (M=Ho) and the amount of added acetone for improved denaturation, all eight cysteines in the protein have reacted.

The nine α-lactalbumin species were observed, from the unlabeled to the completely labeled protein. At a twenty-fold excess of M-MeCAT/cysteine, no further shift of the protein band appeared.

To study migration behavior, a reduced sample of BSA was divided into two aliquots and labeled using M-MeCAT_{Bnz} (M=Tm, Lu). Then different amounts of labeled protein were separated on SDS-PAGE and stained with Coomassie, resulting in only one visible band (FIG. 6B).

To verify that this band contains both MeCAT labels, one of the protein bands was cut out (1 μg), solubilized and subjected to FIA/ICP MS for quantification (FIG. 7A). Two signals of the same area were detected for thulium and lutetium.

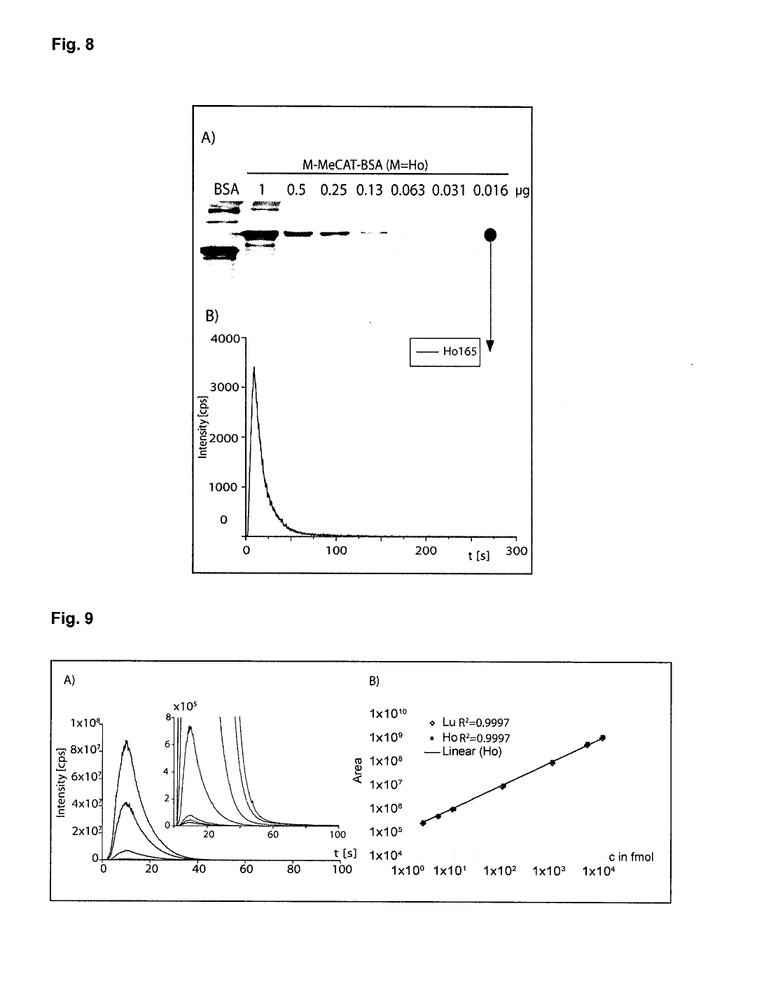

In FIG. 8, the detection limit of ICP MS for protein quantification is shown. To this end, different amounts of labeled BSA reaction mixture (16 ng, 31 ng, 63 ng, 125 ng, 250 ng, 500 ng, 1000 ng) were separated using SDS-PAGE. After silver staining, the protein bands were cut out, solubilized, and diluted for FIA/ICP MS analysis using external calibration (FIG. 9).

When the microconcentric nebulizer was employed, 3.95 fmol of holmium from Ho-MeCAT_{Bnz} labeled BSA were detected. Considering the 35 labels in BSA, 110 amol of this protein were found. For Ho-MeCAT_{Bnz} labeled α-Lactalbumin, 5.37 fmol Holmium could be measured, which accounts for 670 amol α-Lactalbumin, eight-fold labeled.

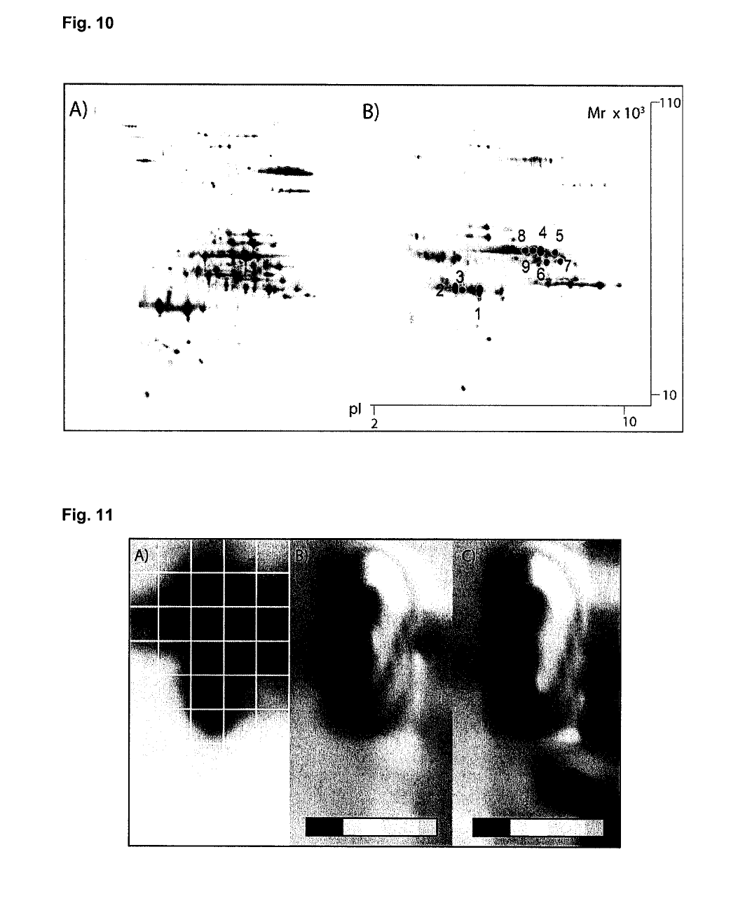

In the next step proteins from the eye lens of Sus scrofa were tagged and analyzed. The proteins were diluted under denaturing conditions, reduced and two samples were labeled using M-MeCAT_{Bnz} (M=Lu, Tm). Then the samples were combined and separated using micro-preparative 2-D electrophoresis (FIG. 10). The Coomassie stained spots were cut out and digested using trypsine for LC/ESI MS analysis. To quantify the proteins using ICP MS, the spots from another silverstained analytical gel were cut out and dissolved (PFA AG, Berlin, Germany).

The solutions were diluted by a factor of twenty-five and analyzed by FIA/ICP MS. In table 2 qualitative data with name of proteins, searched database, accession number, mascot score and sequence coverage obtained from micropreparative 2-D gel are compiled. Spots 1-4 were identified as α-Crystallin A isoforms, spots 5/6 as β-Crystallin B2 and spots 7-9 as β-Crystallin B3 isoforms. Furthermore, table 2 shows quantitative data of the crystallins. The amounts of proteins range from 37 ng up to 970 ng for the Lu-MeCAT labeled sample and from 44 ng to 760 ng for Tm-MeCAT labeled sample. This is equivalent to a dynamic range of 1.5 orders of magnitude.

TABLE 2

| | | | | Mascot | Coverage | n Tm | n Lu | m [g] | m [g] |
|---|---|---|---|---|---|---|---|---|---|
| Spot | Protein | Database | gi No. | score | [%] | [mol] | [mol] | Protein Tm | Protein Lu |
| 1 | Alpha crystallin A chain | Ss EST | 13658496 | 469 | 42 | 3.9E−11 | 4.9E−11 | 7.6E−07 | 9.7E−07 |
| 2 | Alpha crystallin A chain | Ss EST | 13658496 | 443 | 41 | 1.4E−11 | 1.8E−11 | 2.8E−07 | 3.5E−07 |
| 3 | Alpha crystallin A chain | Ss EST | 13658496 | 462 | 57 | 3.3E−12 | 3.3E−12 | 6.4E−08 | 6.5E−08 |
| 4 | Chain C, Beta B2 Crystallin | NCBInr* | 809228 | 517 | 50 | 6.0E−12 | 6.5E−12 | 1.2E−07 | 1.3E−07 |
| 5 | Chain C, Beta B2 Crystallin | NCBInr* | 809228 | 517 | 50 | 3.8E−12 | 3.2E−12 | 4.4E−08 | 3.7E−08 |
| 6 | Crystallin, beta B3 | Ss EST | 46176196 | 543 | 41 | 4.3E−12 | 4.2E−12 | 5.0E−08 | 4.8E−08 |
| 7 | Crystallin, beta B3 | Sc EST | 46176196 | 603 | 43 | 1.5E−11 | 1.7E−11 | 9.5E−08 | 1.1E−07 |
| 8 | Crystallin, beta B3 | NCBInr* | 73994848 | 534 | 29 | 1.7E−11 | 2.2E−11 | 1.8E−07 | 2.4E−07 |
| 9 | Crystallin, beta B3 | Ss EST | 46176196 | 763 | 34 | 1.8E−11 | 2.1E−11 | 2.0E−07 | 2.3E−07 |

To explore the limits of detection further, one spot of this gel was thoroughly analyzed with a spatial resolution of 1 mm. The intensities measured were used to construct a three dimensional profile of the entire spot (see FIG. 11). The data provide evidence for the superior sensitivity of ICP MS allowing detecting the low-level concentrations at the border of the spots. Limitations are due to the capacity of the analytical gel, not to the detection limits of the ICP MS.

Due to the low natural level of lanthanide concentration, the virtually absent background and the detection capability of ICP MS the method according to the invention is sensitive enough for absolute quantification of intact proteins at attomol levels (e.g. BSA 110 attomol). Optimized affinity preconcentration/purification strategies should lead to the identification and quantification of very low abundant proteins.

Then, loss of protein due to sample properties and preparation procedures, unspecific binding and impurities cannot be followed with the quantification methods in use. MECAT should help to monitor each sample handling step thus allowing optimizing the protocols for increased yields.

In addition, a direct comparison of different proteins in complex mixtures is feasible, which gives access to the analysis of protein complexes and protein species.

An unsolved problem in quantitative protein analysis is the dynamic range of concentration in biological systems, covering up to ten orders of magnitude. Here, only ICP MS offers the required dynamic range. We could demonstrate during calibration using metals in dissolved gels a dynamic range of four orders of magnitude. In principle, for ICP MS, up to 12 orders of magnitude have been shown for lanthanide samples. Furthermore, analysis of one spot by FIA/ICP MS is rapid (60 sec per spot) and automation will be implemented. It is evident that MeCAT cannot only be used for absolute, but for relative quantification of four or more samples simultaneously as well. Since absolute determinations are easy to achieve and very accurate, even quite different samples and preparations can be compared, which is another unique aspect of this strategy. The prospect to cover such a dynamic range and the superior sensitivity encourages us to develop the technical parameters necessary to make MeCAT a viable alternative to any other techniques available or under development.

Figure Captions

FIG. 1: Structures of the trifunctional MeCAT reagents used for cycsteine labeling. The macrocycle has been loaded with triply charged lanthanide ions (M). A) MeCAT_{BNZ} consists of (I) the DOTA macrocycle for metal coding, (II) a spacer that connects the macrocycle and the reactive maleimido group (III) for thiol specific labeling. B) Shows MeCAT_{BIO} which in addition carries a biotin affinity group for preconcentration and purification.

FIG. 2: The MeCAT workflow. Proteins from different samples are labeled at the cysteine residues with differently metal coded M-MeCAT reagents. On the way to combining of the samples, the protein mixture could be subjected to any separation method. To monitor the labeling efficiency and reduce the complexity at the protein level, SDS-PAGE or 2-D electrophoresis was used. For absolute quantification, the slices or spots were dissolved and analyzed using FIA/ICP MS. Since the proteins from each sample are modified with a different M-MeCAT label, the metals can be used to trace the proteins solely based on the metal signals in ICP MS. The size of the respective signal reflects for each protein the amount coming from the original sample. Since internal standards can be added absolute and relative quantification is possible. After proteolysis, LC/ESI MSn at peptide level follows for the identification.

FIG. 3: Extracted ion chromatograms (EIC) of two BSA peptides (RPC*FSALTPDEYVPK, LC*VLHEK) labeled with two different M-MeCAT_{Bnz} reagents (M=Ho, Lu). Integration of the signals yields a ratio Ho:Lu of 2:1 reflecting the used amounts of differently labeled peptides.

FIG. 4: CID MS/MS spectrum of a Ho-MeCAT_{Bnz} (*) labeled BSA peptide. 800 pmol of a tryptic BSA digest was analyzed using micro-HPLC/MS/MS. The spectrum shows the y and b series of a doubly protonated precursor ion. In addition, signals of the immonium ion of the labeled Cys, Thiol-Ho-MeCAT_{Bnz}: (HS-MeCAT) and the loss of H2O are visible.

FIG. 5: MALDI-ToF MS spectra of A) fully labeled HIV integrase peptide (HIV II) with four different M-MeCAT_{Bio} reagents (M=Tb, Ho, Tm, Lu) reflecting the M-MeCAT ratios Tb:Ho:Tm:Lu of 2:1:2:1; B) the labeled HIV II, spiked in an E. coli cell lysate digest; the peptides are invisible in the mixture C) streptavidin magnetic beads affinity purification of labeled HIV II spiked E. coli cell lysate digest. The peptides only and a minor quantity of the reagent were recovered and the ratio of the metals remains unchanged.

FIG. 6: Silver stained SDS-PAGE of pure α-Lactalbumin and six labeling mixtures; the excess of M-MeCAT reagent (M=Ho) and the amount of acetone was varied. The visible bands represent the intermediate labeling stages up to the completely labeled α-Lactalbumin (8 MeCAT groups).

FIG. 7: B) Co-migration experiments on SDS-PAGE of M-MeCAT_{Bnz} labeled BSA (M=Lu, Tm) mixed in equal amounts. The marked Spot (1 μμg) was cut out, dissolved, and diluted for FIA/ICP MS analysis (A). Integrated peak areas result in the ratio Lu:Tm of 1:1.

FIG. 8: A) Silver stained SDS-PAGE, loaded with a dilution series of M-MeCAT_{Bnz} labeled BSA (M=Ho). B) ICP MS trace of Ho allows the detection of the lowest amount of labeled protein, not visible in the gel with silver staining.

FIG. 9: A) FIA/ICP MS of Holmium and Lutetium standard solutions, B) Linear calibration functions resulting from measured metal standards. The calibrations functions are identical and remain linear over a wide concentration range, only four orders of magnitude are shown here.

FIG. 10: Silver stained 2-D electrophoresis of Sus scrofa eye lens proteins A) separation of the unmodified proteins, B) separation of a mixture of M-MeCAT_{Bnz} labeled eye lens proteins (M=Lu, Tm), the indicated spots 1-9 were analyzed using FIA/ICP MS

FIG. 11: Detailed analysis of one single spot (Spot 1 of FIG. 10 B). This spot was divided into 45 gel pieces (grid 1 mm×1 mm, (A)); each piece was analyzed for Lu and Tm using FIA/ICP MS. B) shows a reconstructed 3-D surface map for the Tm labeled MeCAT_{Bnz} protein and C) for the same protein, Lu-MeCAT_{Bnz} labeled. The color coding in B) and C) reflect the amount of metal contained in each field ranging from dark grey black to white.

FIG. 12: Approach for quality and product control

FIG. 12: Approach for proteome analysis

1. Method for the identification and quantification of one or more biomolecules in a sample containing a mixture of substances, wherein said method comprises the steps of:
a) providing a sample which contains one or more biomolecules;
b) providing a reagent for the analysis of biomolecules according to Formula I;
A-Y-PRG Formula I
wherein, A may be present or absent and when A is present, A is H or constitutes at least one functional group for the reversible, covalent or non-covalent binding to a support material, whereby Y is a group comprising at least one chelate function for metals, preferable low in isotopes, and whereby PRG is a reactive group for the selective binding of biomolecules to be analyzed;
c) coupling the one or more biomolecules from step a) to the reagent of step b);
d) selecting the one or more biomolecules labeled in step c) under the employment of a functional group for the reversible, covalent or non-covalent binding to a support material and removal of the one or more unbound biomolecules, wherein (d) optionally comprises releasing the bound biomolecules from the support material and the elution from the matrix; and
f) detecting and identifying the one or more labeled biomolecules by means of Inductively Coupled Plasma-Mass Spectrometry (IC PMS), wherein f) optionally comprises cleaving the one or more biomolecules in said sample wherein, said cleaving may be performed (i) before coupling of the reagent PRG or (ii) after coupling.

2. Method according to claim 1, wherein prior to the detecting step f), a fractionation step is included and the means of fractionation is selected from the group of (i) fractionation based on chemical properties of the sample and/or the one or more labeled biomolecules, (ii) physical properties of the sample and/or the one or more labeled biomolecules, (iii) both, chemical and physical properties of the sample and/or the one or more labeled biomolecules.

3. Method according to claim 2, wherein the means of fractionation is selected from the group of HPLC, LC, FPLC, spin column separation, affinity chromatography, size exclusion chromatography, hydrophobic interaction chromatography, ion exchange chromatography, chromatofocussing, immuno precipitation, gel based methods, 2DGE, electrophoresis, breakable PAGE gels, isoelectric focussing, free-flow electrophoresis, capillary electrophoresis and separation by centrifugation or ultracentrifugation.

4. Method according to claim 1, wherein the fractions are further analyzed by additional chemical and biological methods, fractionation, chemical or enzymatical cleavage, fractionation and mass spectrometric methods for quantitation and identification of biomolecules present in the fractions.

5. Method according to claim 1, wherein arrangement of the groups A, X and PRG is interchanged.

6. Method according to claim 1, wherein the one or more biomolecules in the sample are selected from the group of, (a) one or more known biomolecules and wherein the reagent according to Formula I is bound to said one or more known biomolecules, (b) one or more unknown biomolecules and wherein the reagent according to Formula I is bound to said one or more unknown biomolecules.

7. Method according to claim 6, wherein, the known and unknown biomolecule is selected from the group comprising a pyrogen, a pharmaceutical compound, a pharmaceutical lead compound, an allergen, an autoimmunogen, a toxin, a polyclonal antibody, a monoclonal antibody, an antigen, a lipid, a carbohydrate, a peptide, a protein, a protein complex, an amino acid, a fatty acid, a nucleotide DNA, RNA, PNA, siRNA and microRNA.

8. Method according to claim 1, wherein, the one or more biomolecules present are quantified with the aid of an internal standard, wherein the internal standard is a solution containing known metal concentrations of at least one or more metals and/or the internal standard consists of one or more biomolecules of known concentration which are labelled by one or more reagents according to the general Formula I
A-Y-PRG Formula I
whereby, A may be present or absent and when A is present, A is H or constitutes at least one functional group for the reversible, covalent or non-covalent binding to a support material, whereby Y is a group comprising at least one chelate function for metals, preferable low in isotopes, and whereby PRG is a reactive group for the selective binding of biomolecules to be analyzed;

9. Method according to claim 1, wherein the one or more biomolecules are present in the sample at an amount of less than 1 millimol, or less than 1 micromol or less than 1 nanomol, or less than 1 picomol, or less than 1 femtomol, or less than 1 attomol or at an amount less than 1 milligram, or less than 1 microgram or less than 1 nanogram, or less than 1 picogram, or less than 1 femtogram, or less than 1 attogram.

10. Method according to claim 1, wherein the dynamic range of quantities measured in the reaction is higher than 10⁶, higher than 10⁷, higher than 10⁸, higher than 10⁹, higher than 10¹⁰, or higher than 10¹¹.

11. Method according to claim 10, wherein the dynamic range of the quantities of biomolecules analyzed can vary between 10⁰ and 10⁹ in concentration and if two or more biomolecules are analyzed their respective concentrations may vary between 10⁰ and 10⁹ in concentration.

12. The method according to claim 1 for the detection of peptides in a biological sample and/or for determining the relative and/or absolute expression of proteins in a protein-containing sample.

13. The method according to claim 1 for performing quality control assays during or after production processes in the field of chemistry, food production, pharmacy, production of recombinant proteins and peptides, amino acids, carbohydrates, lipids, fatty acids and diagnostic assays.
